(19) United States
(12) Patent Application Publication
(10) Pub. No.: US 2025/0228982 A1
(43) Pub. Date: Jul. 17, 2025

(54) OXIDATIVE NANOPARTICLES FOR USE IN THE TREATMENT OF DISEASES AND MEDICAL CONDITIONS

(71) Applicant: Rodney Herring, Victoria (CA)

(72) Inventor: Rodney Herring, Victoria (CA)

(21) Appl. No.: 19/022,894

(22) Filed: Jan. 15, 2025

(30) Foreign Application Priority Data
Jan. 15, 2024 (CA) 3226232

(51) Int. Cl. A61K 51/02; A61K 51/12

(52) U.S. Cl. CPC A61K 51/025; A61K 51/1244; A61K 2121/00

(57) ABSTRACT

A doped nanoparticle for use in the treatment of a disease or condition in the absence of a light source, the doped nanoparticle comprising: an iron and fluorine 18 doped titanium dioxide nanoparticle or an iron and gallium 68 doped titanium dioxide nanoparticle.

### CROSS REFERENCE TO RELATED APPLICATIONS

The present application claims benefit of Canadian Patent Application Serial No. 3226232 filed Jan. 15, 2024 and entitled OXIDATIVE NANOPARTICLES FOR USE IN THE TREATMENT OF DISEASES AND MEDICAL CONDITIONS, the contents of which are incorporated herein by reference.

### FIELD

The present technology is directed to titanium dioxide nanoparticles and titanium dioxide nanoparticles that are doped with fluorine-18 (F18) or gallium-68 (Ga68) or are co-doped with F18 and iron or co-doped with Ga68 and iron. More specifically, the present technology is F18 or Ga68, low iron oxide, iron doped titanium dioxide and the use thereof in the treatment of diseases and conditions, for examples, cancer and Alzheimer's, in the absence of a light source.

### BACKGROUND

A major medical use of fluorine-18 (F18) is in positron emission tomography (PET) to image the brain and heart, to image the thyroid gland, as a radiotracer to image bones, looking for cancers that have metastasized from other locations in the body and in radiation therapy treating internal tumors. FlutemetamolF18 (C₁₄H₁₁^{F18}N₂OS) is a PET scanning radiopharmaceutical containing the radionuclide fluorine-18, used as a diagnostic tool for Alzheimer's disease. F18 has a half-life of 109 minutes, which is enough time to administer and image objects in the body. It provides high resolution PET image since its emitted positron only travels 2-3 mm from the point of F18 decay. When F18 decays it changes its properties from fluorine, which has a monotonic charge of +1, to oxygen, which has a diatonic charge of +2.

When titanium oxide nanoparticles are consumed, they easily pass through the intestine into the blood stream, can pass through the blood brain barrier and can be absorbed by cells. These nanoparticles are biocompatible and are photo-excitable. These features have led to their use in a number of biomedical applications in which the photo-activation is key. Photoactivated titanium dioxide nanoparticles induce generation of pairs of electrons and holes which react with water and oxygen to yield reactive oxygen species (ROS) that have been proven to damage cancer cells, triggering controlled cellular processes. The requirement for a light source in order to treat a disease is a major limitation to the utility of the approach and the materials.

United States Patent Application Publication No. 20060210798 discloses metal oxide nanoparticles that contain a non-metallic dopant selected from the group consisting of boron, carbon, silicon, germanium, nitrogen, phosphorous, arsenic, sulfur, selenium, tellurium, fluorine, chlorine, bromine, iodine, and combinations thereof. Methods of making and using these doped metal oxide nanoparticles are also described. Methods of use include light activation of the doped nanoparticles in the treatment of cancer. This approach relies upon light to activate the nanoparticles. The role of the dopant is to allow for improved light activation of the titanium dioxide nanoparticles. The requirement for a light source in order to treat a disease is a major limitation to the utility of the approach and the materials.

United States Patent Application Publication No. 20150335744 discloses nanoparticles comprising a metal oxide and a platinum cluster having a height to base ratio greater than 1 and compositions containing the same are disclosed. Methods of using the nanoparticles in producing hydroxyl radicals and in photodynamic therapy, for example, in the treatment of hyperproliferative disease such cancer, are also disclosed. The role of the dopant is to allow for improved light activation of the titanium dioxide nanoparticles. The requirement for a light source in order to treat a disease is a major limitation to the utility of the approach and the materials.

U.S. Pat. No. 7,431,808 discloses an electrically conductive titanium dioxide sputter target with an electrical resistivity of less than 5 Ω-cm, which contains as an additive at least one doping agent or a mixture of doping agents in an amount of less than 5 mole %. The doping agent or agents are selected from the group including indium oxide, zinc oxide, bismuth oxide, aluminum oxide, gallium oxide, antimony oxide, and zirconium oxide. This treatment renders the titanium dioxide sputter target suitable for use in a direct-current sputtering process without any negative effects on the properties of the coating.

What is needed are F18 or Ga68 doped titanium dioxide nanoparticles for concomitantly scanning and treating diseases in the absence of light. It would be preferable if the nanoparticles were suitable for PET scanning. It would be preferable if the nanoparticles could be used to treat cancer or Alzheimer's. It would be still preferable if the nanoparticles could be conjugated to targeting materials including small molecules and monoclonal antibodies for use in the absence of PET scanning. It would be preferable if the nanoparticles were further doped with iron.

### SUMMARY

Provided are F18 or Ga68 doped titanium dioxide nanoparticles for concomitantly scanning and treating diseases. The nanoparticles are suitable for PET scanning. The nanoparticles can be used to treat cancer or Alzheimer's in the absence of light. The nanoparticles can be conjugated to targeting materials including small molecules and monoclonal antibodies for use in the absence of PET scanning. The nanoparticles are further doped with iron. The nanoparticles are further doped with iron and acid washed to reduce or eliminate iron oxide on the surface of the nanoparticles. The iron doped, acid washed nanoparticles provide an additional advantage by reducing the recombination of the electron/hole pairs.

In one embodiment, a doped nanoparticle for use in the treatment of a disease or condition in the absence of a light source, the doped nanoparticle comprising: titanium dioxide nanoparticles, the titanium dioxide nanoparticle including interstitial spaces between oxygen atoms and titanium atoms; and fluorine 18, the fluorine 18 located in at least the interstitial spaces.

In the doped nanoparticle, the titanium dioxide nanoparticle may further comprise iron to provide a fluorine 18, iron doped titanium dioxide nanoparticle.

In the doped nanoparticle, the fluorine 18, iron doped titanium dioxide nanoparticle includes a surface, and the surface may be less than 5% iron oxide.

In another embodiment, a method of synthesizing a doped nanoparticle is provided for the treatment of a disease or a condition in the absence of a light source, the method comprising doping titanium dioxide nanocrystals with iron to provide an iron-doped titanium dioxide nanoparticle, drying the iron-doped titanium dioxide nanoparticle to provide a dried iron-doped titanium dioxide nanoparticle powder, wherein the doping and the drying is prepared by a sol-gel method using titanium isopropoxide as a precursor and ferric nitrate as an iron source, and doping the dried iron-doped titanium dioxide nanoparticle powder with fluorine 18.

The method may further comprise washing the dried iron-doped titanium dioxide nanoparticle powder with an acidic solution having a pH of no higher than about 4, and rinsing with water, prior to doping with fluorine 18.

In another embodiment, use of a doped nanoparticle is provided for the detection of a disease in the absence of a light source, the method comprising selecting a titanium oxide nanoparticle, the titanium oxide nanoparticle including fluorine 18; administering the doped nanoparticle to a patient in need thereof; and the fluorine 18 decaying thereby activating the doped nanoparticle in the absence of a light source.

In the use the titanium oxide nanoparticle may further include iron.

The use of claim may further comprise concomitantly treating the disease.

In the use, the disease may be cancer.

In the use, the disease may be Alzheimer's.

In another embodiment, a combination is provided for use in the treatment of a disease or condition in the absence of a light source, the combination comprising a titanium oxide nanoparticle and flutemetamol F18, wherein the titanium oxide nanoparticle and flutemetamol F18 are tagged to one another.

In the combination, the titanium oxide nanoparticle includes iron.

In the combination, the titanium oxide nanoparticle may include a surface with less than 5% iron oxide.

In another embodiment, a doped nanoparticle is provided for use in the treatment of a disease or condition in the absence of a light source, the doped nanoparticle comprising: an iron and fluorine 18 doped titanium dioxide nanoparticle or an iron and gallium 68 doped titanium dioxide nanoparticle.

In the doped nanoparticle, the iron and fluorine 18 doped titanium dioxide nanoparticle and the iron and gallium 68 doped titanium dioxide nanoparticle may include a surface, and the surface is less than 5% iron oxide.

In the doped nanoparticle, the iron and fluorine 18 doped titanium dioxide nanoparticle and the iron and gallium 68 doped titanium dioxide nanoparticle may contain between 10 atomic percent to 16 atomic percent fluorine 18 or gallium 68, respectively.

The doped nanoparticle may be the iron and fluorine 18 doped titanium dioxide nanoparticle.

The doped nanoparticle may be the iron and gallium 68 doped titanium dioxide nanoparticle.

In another embodiment, provided is a method of treating a disease or a condition in a patient in need thereof, in the absence of a light source, the method comprising: administering a dose of iron and fluorine 18 doped titanium dioxide nanoparticles or a dose of iron and gallium 68 doped titanium dioxide nanoparticles to the patient, wherein the nanoparticles include a surface and the surface is less than 5% iron oxide; and activating the nanoparticles, thereby treating a disease or condition in the patient.

In the method, the dose may be 200 to 300 milligrams of the nanoparticles.

In the method, the administering may be oral administration.

In the method, the iron and fluorine 18 doped titanium dioxide nanoparticles may be administered to the patient.

In the method, the activating may be by fluorine 18 decay.

In the method, the iron and gallium 68 doped titanium dioxide nanoparticles may be administered to the patient.

In the method, the activation may be by gallium 68 decay.

In the method, the activation may be with proton beams.

In the method, the activation may be with X-rays.

In the method, the activation may be with proton beams.

In the method, the activation may be with X-rays.

In the method, the disease may be cancer.

In the method, the disease may be Alzheimer's.

In the method, the disease may be cancer.

In the method, the disease may be Alzheimer's.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Low iron oxide, iron-doped titanium dioxide-in the context of the present technology, low iron oxide, iron-doped titanium dioxide has about 0.1 atomic % iron to about 2.0 atomic % iron, preferably 0.25 atomic % iron to about 0.75 atomic % iron, and more preferably 0.5 atomic % iron and very small amounts of iron oxide on its surface (less than 5% of the surface being iron oxide) when viewed with X-ray photoelectron spectroscopy as disclosed in U.S. Pat. No. 10,413,888.

Substantially iron oxide free-in the context of the present technology, a substantially iron oxide free surface has an iron oxide content corresponding to less than about 0.001 atomic % iron (less than 0.5% of the surface being iron oxide) when viewed with X-ray photoelectron spectroscopy as disclosed in U.S. Pat. No. 10,413,888.

### DETAILED DESCRIPTION

In one embodiment, F18 was used to dope titanium dioxide nanoparticles. In another embodiment, F18 was used to dope low iron oxide, iron doped titanium dioxide nanoparticles. In another embodiment, F18 was used to dope iron doped titanium dioxide nanoparticles. In yet another embodiment, F18 and iron were used to co-dope titanium dioxide nanoparticles. In all the embodiments, this resulted in between 10 atomic % and 16 atomic % F18 in the nanoparticles. The F18 was found in the titanium dioxide nanoparticles within the interstitial sites, i.e., between the titanium and the oxygen atoms. All the embodiments were used to tag flutemetamol F18.

In one embodiment, Ga68 was used to dope titanium dioxide nanoparticles. In another embodiment, Ga68 was used to dope low iron oxide, iron doped titanium dioxide nanoparticles. In another embodiment, Ga68 was used to dope iron doped titanium dioxide nanoparticles. In yet another embodiment, Ga68 and iron were used to co-dope titanium dioxide nanoparticles. In all the embodiments, this resulted in between 10 atomic % and 16 atomic % Ga68 in the nanoparticles. In Ga68 doped titanium dioxide nanoparticles there was about atomic % to 16 atomic % Ga68 in the nanoparticles. The Ga68 was found to replace titanium in the titanium dioxide nanoparticles.

The various embodiments of F18 and Ga68 doped titanium dioxide nanoparticles and F18 and Ga68 low iron oxide, iron co-doped titanium dioxide nanoparticles, as described above were synthesized for use in PET scanning for diseases and other medical conditions. One method of doping is adsorbing F18 or Ga68 on the titanium dioxide nanoparticles or the low iron oxide, iron doped titanium dioxide nanoparticles.

Without being bound to theory, F18 in F18 doped titanium dioxide nanoparticles decays to oxygen. The oxygen removes one of the electrons from titanium dioxide, which then creates a positive charge or positive hole in the titanium dioxide. The positive hole can percolate through the titanium dioxide nanoparticle to its surface where it can create oxidants such as hydroxyl radicals from the hydroxide ions present in the pH 7.3 blood and within the cytoplasm of the cell.

It is proposed that the titanium dioxide in the various F18 doped nanoparticles absorb the 511 keV photons, creating as many as 511,000 eV/3 eV=170,333 pairs of electrons and holes, many of which would recombine but many would reach the surface to create hydroxyl radicals and/or super oxide radicals and/or other activated oxygen species. The F18 doped, iron doped titanium dioxide nanoparticles, the F18 doped, low iron oxide, iron doped titanium dioxide nanoparticles or the co-doped F18, iron doped titanium dioxide add an additional advantage by reducing the recombination of the electron/hole pairs. Even if the nanoparticles only absorbed one of the two photons, 85,167 pairs of electrons and holes would be generated.

It is further proposed that F18 doped TiO2 captures the positron emitted by F18's decay, which has 633 keV energy, i.e., 633,000 eV energy. This is captured by the various embodiments of F18 doped titanium dioxide nanoparticles. Additionally, within various embodiments of F18 doped titanium dioxide nanoparticles is a positive vacancy, which maintains neutrality of the various embodiments of F18 doped titanium dioxide nanoparticles. Having 16 atomic % F18 means there will be 16% positive vacancies produced by the time F18 is depleted, which translates to a high amount of hydroxyl radicals and/or super oxide radicals and/or other activated oxygen species. For 16 atomic % of a 100 nanometer diameter F18 doped titanium dioxide nanoparticle there are 4.56×10exp (6) hydroxyl radicals (titanium dioxide density of 3.78 g/cm3 or 3.78×10exp (6) g/m3, volume of (100 nm×10exp (−9))*3/m3, Avogadro's no.=6.022×10exp (23) atoms/mole and molecular weight of 79.866 g/mole). Thus, the emitted positron from F18 will see a large positive potential within the various embodiments of F18 doped titanium dioxide nanoparticles, which will assist the various embodiments of F18 doped titanium dioxide nanoparticles capture the positron. There will be 633,000 eV/3 eV (the bandgap of TiO2)=211,000 positive charges within the titanium dioxide nanoparticle. Since no free electrons are being created by the capture of the positron within the various embodiments of F18 doped titanium dioxide nanoparticles, there will be little or no annihilation mechanism between the electrons and holes to annihilate these positive charges.

The various embodiments of F18 doped titanium dioxide nanoparticles were used during PET scanning and reduced or eliminated the cancer cells being imaged. The dosage was about 200 to 300 milligrams per dose. The dose was administered orally. In an alternative embodiment, the dose was delivered intravenously.

The various embodiments of Ga68 doped titanium dioxide nanoparticles were used during PET scanning and reduced or eliminated the cancer cells being imaged. The dosage was about 200 to 300 milligrams per dose. The dose was administered orally. In an alternative embodiment, the dose was delivered intravenously.

The various embodiments of F18 doped titanium dioxide nanoparticles, as described above, were tagged to flutemetamol for PET imaging of amyloid beta plaque. F18 in F18 doped titanium dioxide nanoparticles decays to oxygen. The oxygen removes one of the electrons from titanium dioxide, which then creates a positive charge or positive hole in the titanium dioxide. The positive hole can percolate through the titanium dioxide nanoparticle to its surface where it can create oxidants such as hydroxyl radicals and/or super oxide radicals and/or other activated oxygen species. As the F18 doped titanium dioxide nanoparticles can pass through the blood brain barrier, the oxidants degraded the amyloid plaques during imaging.

In another embodiment, F18-glucose was administered while titanium dioxide nanoparticles, iron doped titanium dioxide nanoparticles or low iron oxide, iron doped titanium dioxide nanoparticles were also administered. The two in concert reduced or eliminated the cancer cells, or amyloid beta plaques. The mode of action is proposed to be via production of photons as F18 decays. The photons strike the titanium dioxide nanoparticles, iron doped titanium dioxide nanoparticles or low iron oxide, iron doped titanium dioxide nanoparticles leading to the production of hydroxyl radicals and/or super oxide and/or other activated oxygen species from the titanium dioxide nanoparticles, iron doped titanium dioxide nanoparticles or low iron oxide, iron doped titanium dioxide nanoparticles. The efficiency is expected to be low since the capture of the high energy photon relies upon the photo-electric effect, which has very low efficiency on the order of 1 photon captured per 10exp (25) photons emitted.

In another embodiment, the various F18 doped titanium dioxide nanoparticles were conjugated to molecules known to target specific tissues or conditions for example, but not limited to, small molecules and monoclonal antibodies for the treatment of cancers.

In another embodiment, Ga68-glucose was administered while titanium dioxide nanoparticles, iron doped titanium dioxide nanoparticles or low iron oxide iron doped titanium dioxide nanoparticles were also administered. The two in concert reduced or eliminated the cancer cells, or amyloid beta plaques. The mode of action is proposed to be via production of photons as Ga68 decays. The photons strike the titanium dioxide nanoparticles, iron doped titanium dioxide nanoparticles or low iron oxide, iron doped titanium dioxide nanoparticles leading to the production of hydroxyl radicals and/or super oxide and/or other activated oxygen species from the titanium dioxide nanoparticles, iron doped titanium dioxide nanoparticles or low iron oxide, iron doped titanium dioxide nanoparticles. The efficiency is expected to be low since the capture of the high energy photon relies upon the photo-electric effect, which has very low efficiency on the order of 1 photon captured per 10exp (25) photons emitted.

In another embodiment, the various Ga68 doped titanium dioxide nanoparticles were conjugated to molecules known to target specific tissues or conditions for example, but not limited to, small molecules and monoclonal antibodies for the treatment of cancers.

The low iron oxide iron doped titanium dioxide nanoparticle was prepared by the sol-gel method using titanium isopropoxide (TTIP) as the precursor and ferric nitrate (Fe(N0₃)3·9H₂0) as the iron source. Firstly, the desired amount of ferric nitrate (0.25, 0.5, 1, 5 and 10 molar %) was dissolved in water and then the solution was added to 30 mL of anhydrous ethyl alcohol and stirred for 10 minutes. The acidity of the solution was adjusted to about pH 3 (about pH 2.5 to about pH 3.5) using HNO3 (other acids could also be used), which produces better Fe doped Ti0₂, i.e., incorporation of Fe into the Ti0₂ nanocrystals. Secondly, TTIP was added dropwise to the solution. Then deionized water with the ratio of Ti:H₂0 (1:4) was added to the mixture. The solution was stirred for two hours and then dried at 80° C. for two hours. Less than about 5% of the nanoparticle surface was iron oxide.

The powders were then washed three times with deionized water. Next, the powder was calcined at 400° C. for three hours. The calcined powder was then stirred in a solution of about pH 2.5 to about pH 3.5, or about pH 4, with, preferably, a monoprotic acid, such as, for example, but not limited to acetic acid (CH₃CO₂H or HOAc), hydrochloric acid (HCl), hydroiodic acid (HI), hydrobromic acid (HBr), perchloric acid (HClO₄), nitric acid (HNO3) or sulfuric acid (H₂SO₄), with HCl being the preferred and was then washed with deionized water three times.

The iron doped titanium dioxide nanoparticles were prepared by the sol-gel method using titanium isopropoxide (TTIP) as the precursor. TTIP was added to 30 mL of anhydrous ethyl alcohol and stirred for 10 minutes. The solution was stirred for two hours and then dried at 80° C. for two hours. The powders were then washed three times with deionized water. Next, the powder was calcined at 400° C. for three hours.

The titanium dioxide nanoparticles, the iron doped titanium dioxide nanoparticles and the low iron oxide, iron doped titanium dioxide nanoparticles were doped with F18 by plasma doping. The titanium dioxide nanoparticles, the iron doped titanium dioxide nanoparticles or the low iron oxide, iron doped titanium dioxide nanoparticles were placed in a plasma furnace and bombarded with F18. As this is a rapid process the products can be produced within the timeframe needed for PET imaging.

In another embodiment, cyclotron irradiation was used to make F18 from O18, which was done on any of the titanium dioxide nanoparticles (titanium dioxide nanoparticles, the iron doped titanium dioxide nanoparticles and the low iron oxide, iron doped titanium dioxide nanoparticles). This made doping by F18 at the same time as making F18 from O18.

In other embodiments, the titanium dioxide nanoparticles or the iron doped titanium dioxide nanoparticles or the low iron oxide iron doped titanium dioxide nanoparticles (each of which is a separate embodiment) is synthesized with O 18 instead of 016. These nanoparticles were converted in the cyclotron to titanium difluoride 18. These nanoparticles are taken up by the cancer cells and decay to titanium dioxide-18 where every decay from F18 to O18 produces a positive charge in the nanoparticle which is consumed and makes a hydroxyl radical within the cancer cell. The number of hydroxyl radicals for a 100 nm×100 nm×100 nm nanoparticle increases to 1,000,000 as compared to 100,000 when 10 atomic % F18 was used to dope the nanoparticles. was infused into TiO2 as previously described. Thus, the potency is increased by 10 times.

In an alternative embodiment, pure titanium dioxide nanoparticles were employed.

In yet another embodiment, nitrogen doped titanium dioxide nanoparticles were employed.

In yet another embodiment, transition metals other than iron were used to dope titanium dioxide nanoparticles, for example, but not limited to copper, cobalt and nickel.

Regardless of the type of titanium dioxide nanoparticles (doped with F18 or Ga68 or co-doped with F18 and iron or Ga68 and iron) the nanoparticles may be activated within the patient's body by decay of F18 or Ga68. Without being bound to theory, positron emission and electron capture occur as F18 decays to O18 and Ga68 decays to Zn68. The decay leads to gamma rays and hydroxyl radicals. The decay of F18 to O18 and the decay of Ga68 to Zn68 leads to a one-to-one generation rate of hydroxyl radicals. Without be bound to theory, the high efficiency is due to the absence of recombination of the positive charge created within the nanoparticles because there's no electron created. This means that the positive charges have a high probability of reaching the surface of the nanoparticles and reacting with its surroundings. Additionally, all of the F18 and Ga68 are consumed within an hour or two so there's a flood of hydroxyl radicals within the cancer cell providing a high probability of lysing.

Regardless of the type of titanium dioxide nanoparticles (doped, co-doped or not doped) the nanoparticles may be activated within the patient's body using X-rays. This allows for using the X-rays in both imaging and treating a patient, especially with regard to tumours. In the case of the low iron oxide, iron doped titanium dioxide nanoparticles, Fe+2 and Fe+4 ions are created from Fe+3. This is the same reaction that occurs when the low iron oxide, iron doped titanium dioxide nanoparticles are exposed to light.

In another embodiment, the titanium dioxide nanoparticles (doped, co-doped or not doped) are activated with proton beams.

In an alternative embodiment, the nanoparticles are activated in the patient with a device that passes an electric current of ˜1 milliamperes. This is more specific to treating brain tumours but could be adapted for the treatment of tumours in other regions of the patient's body.

In another embodiment, 2.8 volts is applied to the patient to activate the iron doped titanium dioxide. This may be in pulses, for example, 22 pulses of 1.3 volts.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A doped nanoparticle for use in the treatment of a disease or condition in the absence of a light source, the doped nanoparticle comprising: an iron and fluorine 18 doped titanium dioxide nanoparticle or an iron and gallium 68 doped titanium dioxide nanoparticle.

2. The doped nanoparticle of claim 1, wherein the iron and fluorine 18 doped titanium dioxide nanoparticle and the iron and gallium 68 doped titanium dioxide nanoparticle include a surface, and the surface is less than 5% iron oxide.

3. The doped nanoparticle of claim 2, wherein the iron and fluorine 18 doped titanium dioxide nanoparticle and the iron and gallium 68 doped titanium dioxide nanoparticle contain between 10 atomic percent to 16 atomic percent fluorine 18 or gallium 68, respectively.

4. The doped nanoparticle of claim 3, wherein the doped nanoparticle is the iron and fluorine 18 doped titanium dioxide nanoparticle.

5. The doped nanoparticle of claim 3, wherein the doped nanoparticle is the iron and gallium 68 doped titanium dioxide nanoparticle.

6. A method of treating a disease or a condition in a patient in need thereof, in the absence of a light source, the method comprising: administering a dose of iron and fluorine 18 doped titanium dioxide nanoparticles or a dose of iron and gallium 68 doped titanium dioxide nanoparticles to the patient, wherein the nanoparticles include a surface and the surface is less than 5% iron oxide; and activating the nanoparticles, thereby treating a disease or condition in the patient.

7. The method of claim 6, wherein the dose is 200 to 300 milligrams of the nanoparticles.

8. The method of claim 7, wherein the administering is oral administration.

9. The method of claim 8, wherein the iron and fluorine 18 doped titanium dioxide nanoparticles are administered to the patient.

10. The method of claim 9, wherein the activating is by fluorine 18 decay.

11. The method of claim 8, wherein the iron and gallium 68 doped titanium dioxide nanoparticles are administered to the patient.

12. The method of claim 11, wherein the activation is by gallium 68 decay.

13. The method of claim 9, wherein the activation is with proton beams.

14. The method of claim 9, wherein the activation is with X-rays.

15. The method of claim 11, wherein the activation is with proton beams.

16. The method of claim 11, wherein the activation is with X-rays.

17. The method of claim 9, wherein the disease is cancer.

18. The method of claim 9, wherein the disease is Alzheimer's.

19. The method of claim 11, wherein the disease is cancer.

20. The use of claim 11, wherein the disease is Alzheimer's.
